(19) United States
(12) Patent Application Publication
(10) Pub. No.: US 2012/0010582 A1
(43) Pub. Date: Jan. 12, 2012

(54) LOW-PROFILE WASHABLE GARMENTS FOR CAPTURING BODILY FLUIDS

(75) Inventor: Angela Newnam, Washington, DC (US)

(73) Assignee: Angle Enterprises, L.L.C., Washington, DC (US)

(21) Appl. No.: 12/831,738

(22) Filed: Jul. 7, 2010

(51) Int. Cl. A61L 15/46; A41B 9/04; A41D 13/02; A41D 13/00; A41D 1/06; A61F 13/15; A41C 3/00

(52) U.S. Cl. 604/360; 604/385.15; 604/377; 2/406; 450/1; 2/69; 2/228; 2/227; 2/46

(57) ABSTRACT

Embodiments of the present invention provide an improved garment for capturing bodily fluids. In particular, embodiments of the present invention provide a garment including a main garment body configured to be worn by a wearer and a capturing panel having a receiving surface configured to receive bodily fluids excreted from the wearer attached to the main garment body such that the capturing panel covers at least a first portion of said main garment body. The capturing panel is made out of a natural material, has a thickness no greater than approximately 4 mm, and is configured to transfer bodily fluid received from the wearer away from the receiving surface. A fluid resistant treatment may be applied to at least a second portion of the main garment body, which second portion may be identical to the first portion. A odor-neutralizing treatment may be applied at least a portion of the capturing panel.

### FIELD

The invention generally relates to the field of garments, and more particularly, embodiments of the present invention relate to improved washable garments for capturing bodily fluids.

### BACKGROUND

Millions of people suffer from involuntary excretions of bodily fluid, including urine, menstrual fluid, vaginal discharge, sweat, and breast milk. In the case of involuntary excretions of urine, those individuals that suffer from mild to moderate incontinence, problems which cause the release of 0.5 to more than 2.5 fluid ounces of urine in a single instance, have an abundance of products available to them to address these problems. For example, there are numerous disposable pads, diapers and disposable and non-disposable absorbent undergarments on the market today that are designed to absorb more than 0.5 fluid ounces of urine and keep the wearer of the product and his or her clothing dry. These products generally include at least the following: a top layer designed to allow fluid to pass through but remain relatively dry, a thick, expandable middle core designed to absorb and/or encapsulate the fluid, and a bottom layer designed to prevent leakage of fluid out of the core. While these products provide necessary protection for those suffering from incontinence, for the reasons set forth below, they are not a suitable solution for the millions of people that suffer from minor and occasional leaks and drips of fluid, for example, during exercise, laughing, coughing, pregnancy, or menstruation.

It has been estimated that as many as 1 in 3 women experience occasional drips and leaks of urine amounting to less than a teaspoon of urine in a single instance (as used herein, a teaspoon (abbreviated “tsp.”) is a measurement of volume equaling ⅙ of a fluid ounce), and would benefit from additional protection beyond that provided by the average undergarment. In addition, men not suffering from prostate conditions or mild incontinence also often experience small leaks and drips that are not addressed in daily-wear undergarments. Such minor excretions (the typical leak or drip is only 0.5 tsp. or less) do not require the high level of protection provided by the incontinence products of the prior art. Indeed, due to the numerous layers of such products, and in particular, the thick core that provides the absorption component, these products are bulky and uncomfortable to wear. In many of the products, the absorbent component is configured to expand as it receives and traps urine; indeed, some pads will grow to be more than twice their original thickness. Furthermore, because urine is trapped within the product and not allowed to escape, even if the wearer is only leaking a small amount or urine from time to time, the leaked urine will accumulate within the product, causing the product to grow in size and weight, and forcing the wearer to carry around the entrapped urine and suffer the added discomfort of additional weight and thickness until he or she is able to replace it. Not only is this extremely uncomfortable for the wearer, but it also has the additional problems of being attended by a lingering unpleasant odor and preventing the wearer from being able to comfortably wear clothing that clings to the body for fear that the bulkiness will be visible to others.

In addition to the foregoing drawbacks of the products of the prior art, because the products are constructed to accommodate bulky absorbent components and the weight attributable to entrapped leaked urine, the products do not allow wearers to comfortably wear low-coverage undergarments. Absorbent pads must be placed into large, maximum-coverage undergarments that are capable of sufficiently containing them, while diapers and absorbent undergarments are likewise uniformly large in order to adequately address the incontinence of the wearer. Thus, a woman who prefers thong-style or low-rise bikini underwear is often forced to choose between either wearing her preferred underwear, which would risk leakage to her outer clothing, or wearing a cumbersome and unattractive garment that would ensure that all leaks are prevented. Finally, because the products of the prior art are generally made out of synthetic materials and are designed to trap excreted fluid such that it cannot escape, the ability for air to travel through such products is severely hindered. The lack of breathability can cause moisture to accumulate on the body of the wearer that does not pass through to the absorbent core, leading to discomfort, chafing, unpleasant odors, and the accumulation of bacteria leading to infection.

Because the available incontinence products are unsuitable for treating minor excretions, women experiencing these small drips and leaks of urine often choose to wear a disposable panty liner in their undergarments, despite the fact that such liners are generally intended to address menstrual fluid. While these liners offer some additional protection over the undergarment alone, the protection afforded generally does not rise to the level required by a wearer experiencing leaks of between approximately 0.25 tsp to approximately 0.75 tsp occasionally throughout the day. As with the incontinence products, these liners will reach capacity as they trap more and more leaks over time, causing the wearer to experience the wetness and discomfort associated with a saturated liner. Thus, in order to use these liners comfortably, it is necessary to periodically change out a used liner for a fresh one, an inconvenient and wasteful practice. Moreover, liners, like the thicker pads and incontinence undergarment products, will not offer adequate breathability to the wearer, increasing discomfort of the wearer and the possibility of bacterial infection.

Finally, in addition to the discomfort and embarrassment caused by the thickness, large overall size, and lack of breathability of the prior art products, the actual and social costs of purchasing such products also demonstrate why such products are unsuitable for minor leaks. The majority of available products are disposable or not easily washable, and purchasing such products on a regular basis is expensive and generates excess trash. Furthermore, a social stigma is attached to the purchase and use of incontinence products. Persons not requiring a high level of protection against incontinence should not have to suffer the foregoing costs.

In addition to the absorbent products and liners of the prior art, there are currently some washable undergarment products on the market that are designed to wick moisture away from the body. These products utilize synthetic fibers to achieve their wicking properties, and generally do not offer any sort of protection against leakage to outer clothing. Doctors broadly recommend undergarments made from natural fibers (such as cotton undergarments) due to their superior breathability and hypoallergenic nature. Because of the tendency of synthetic fabrics to cause irritation and infection, many people cannot wear synthetics, especially in undergarments, and must have natural fabrics against the skin.

With regard to persons having overactive sweat glands, garments currently available to address such a condition have wicking and quick-dry properties that provide some assistance in moving the sweat from the body to the outer surface of the garment and increasing the speed with which it evaporates from the garment into the air. While this is helpful when a person does not mind having the exterior surface of their clothing wet, for example, when exercising, it does not help persons who are sweating in casual or more formal clothing and do not desire the wetness to show. Indeed, if wicking and/or quick-dry materials are used in undergarments, such as undershirts or underwear, such materials do nothing to prevent the outer clothing of the wearer from becoming wet.

Thus, there remains a need for washable, low-profile, low-coverage if desired, breathable, quick-drying, odor-eliminating protective garments constructed from natural fabric and capable of capturing small amounts of bodily fluids excreted or secreted from the body so that the wearer feels dry and the wearer's other clothing is protected.

### BRIEF SUMMARY OF EMBODIMENTS OF THE INVENTION

Embodiments of the present invention solve the above-described problem and/or other problems by providing an improved washable garment for capturing bodily fluids. Generally, embodiments of the present invention provide a garment that utilizes a capturing panel sewn into or onto the main body of the garment that is configured to receive small amounts of bodily fluids excreted from the wearer of the garment, including urine, menstrual fluid, vaginal discharge, sweat, breast milk, saliva, gastroesophageal reflux, blood, pus, etc. Where the bodily fluid is excreted into the interior of the garment, as is the case, for example, where the bodily fluid is urine and the garment is underwear, the capturing panel is sewn into the interior crotch portion of the underwear such that it can receive the urine that is excreted. On the other hand, where the bodily fluid is excreted onto the exterior of the garment, as is the case, for example, where the bodily fluid is gastroesophageal reflux and the garment is a baby bib, the capturing panel is sewn onto the exterior surface of the bib such that it can receive the reflux.

Unlike the bulky absorbent pads, thin panty liners, or multi-layer washable incontinence garments of the prior art, in some embodiments, the capturing panel is constructed of a single layer of a thin breathable fabric made from natural fibers that comprises a combination of both wicking fibers and absorbing fibers, for example, a treated cotton fabric. The wicking properties of the capturing panel, combined with the breathable natural fabric, promote wearer comfort and hygiene by keeping the surface of the capturing panel in contact with the wearer relatively dry and allowing air to circulate through the garment and reach the wearer. Indeed, the wicking properties of the fabric cause moisture to be transferred through the thickness of the capturing panel at a faster rate and across a greater surface area of the capturing panel than such moisture would be transferred in the absence of such wicking properties. The absorbing properties of the capturing panel offer suitable protection against leakages of the bodily fluid out of the capturing panel. According to some embodiments, the single-layer capturing panel can have any surface area as long as the area is large enough both to cover that portion of the garment that receives bodily fluid from the wearer and to comfortably hold the maximum amount of bodily fluid that will be received from the wearer in that location in a single instance, but the capturing panel is no more than 4 mm thick, allowing the constructed garment to maintain a low profile against the wearer. Indeed, the addition of the capturing panel to the main body is so subtle due to its minimum thickness that the constructed garment is indiscernible from other daily-wear garments.

Even though the capturing panel may not be constructed of a highly absorbent material capable of holding a large volume of fluid, the combined wicking and absorbing properties of the material allow the capturing panel to be uniquely well suited to capture a small amount of bodily fluid excreted from the wearer without allowing leakage and while promoting fast drying and increasing the comfort of the wearer. According to some embodiments, the wicking fibers are concentrated at the receiving surface of the capturing panel and the absorbing fibers are concentrated at the back surface of the capturing panel, causing moisture to travel through the capturing panel from the receiving surface to the back surface. Furthermore, in some embodiments, additional strategically placed wicking fibers are located throughout the fabric of the capturing panel, including throughout the back surface of the capturing panel, such that any moisture is dispersed across the full area of the back surface of the capturing panel. Therefore, unless the capturing panel is fully saturated, the bodily fluid received by the capturing panel in a particular location is quickly drawn away from the body toward the back surface of the capturing panel where it is broadly dispersed into the absorbing fibers located throughout the capturing panel. This advantageously prevents localized saturation within the capturing panel that would cause leakage out of the capturing panel while also promoting faster evaporation of the fluid in the capturing panel, leading to increased drying times. Thus, the surface of the capturing panel will almost immediately feel drier to the wearer than it would if a non-wicking material was used, and the capturing panel will dry completely in less time, both of which attributes increase the comfort of the wearer.

In addition to including the capturing panel, garments in accordance with embodiments of the present invention may be treated with a water-resistant treatment and an odor-neutralizing treatment. For example, in some embodiments, a durable water repellant treatment is applied to at least a portion of the main body of the garment that is covered by the capturing panel. Thus, in the event any bodily fluid does escape the capturing panel, the water-resistant treatment will prevent such fluid from penetrating the main body portion of the garment and, in the event the garment is an undergarment, eventually reaching the outer garments of the wearer. In some embodiments, the water-resistant treatment maintains the breathability of the main body portion, such that air and water vapor can pass through the main body portion, allowing the capturing panel to dry through evaporation of the captured bodily fluid. Such breathability offers a distinct advantage in terms of comfort and hygiene over the non-breathable waterproofing used in the products of the prior art, as the wearer will remain dry and will not be forced to carry encapsulated body fluid against his or her body until he or she is able to change. According to some embodiments, an odor-neutralizing treatment may be applied to any portion of either the capturing panel or the main body of the garment in order to neutralize, absorb, and/or eliminate any odors that may be attendant to the bodily fluid captured by the capturing panel.

Thus, embodiments of the present invention generally provide garments that effectively capture small amounts of bodily fluid excreted by the wearer, while still offering comfort, subtlety, breathability, and attractiveness to the wearer.

In particular, one embodiment of the present invention provides a washable garment having a main garment body configured to be worn by a wearer and a capturing panel having a receiving surface configured to receive bodily fluids excreted from the wearer attached to said main garment body such that said capturing panel covers at least a first portion of said main garment body, wherein the capturing panel comprises a natural material, has a thickness no greater than approximately 4 mm, and is configured to transfer bodily fluid received from the wearer away from the receiving surface. In some embodiments, the capturing panel has a backing surface configured to interface with the main garment body and the capturing panel is constructed out of a wicking fabric configured to transfer received bodily fluid from the receiving surface to the backing surface. The wicking fabric may include a combination of wicking fibers and absorbing fibers, wherein the wicking fibers are concentrated about the receiving surface and the absorbing fibers are concentrated about the backing surface. In some embodiments, the natural material comprises plant or cellulose fibers, such as a 100% cotton fabric. According to some embodiments, the capturing panel is attached to the main garment body by stitching only at substantially the perimeter of said capturing panel.

In some embodiments of the present invention, a fluid-resistant treatment is disposed on at least a second portion of the main garment body. The second portion of the main garment body may comprise the first portion of the main garment body, or it may be the same as the first portion of the main garment body. In some embodiments, the fluid-resistant treatment does not substantially reduce the breathability of the main garment body. The fluid-resistant treatment may comprise a nanoparticle C6 fluorochemical treatment configured to reduce the ability of fluids and substances to adhere to or penetrate the main garment body.

In some embodiments, an odor-neutralizing treatment is applied to at least a portion of said capturing panel. According to some embodiments, the capturing panel comprises plant or cellulose fibers and the odor neutralizing treatment comprises bonding an odor-encapsulating agent to the capturing panel, reacting a cyclodextrin with a cross-linking agent capable of forming ether bonds with the cyclodextrin and with the capturing panel, and curing the textile material treated with a mixture of the cyclodextrin and the cross-linking agent

According to some embodiments of the present invention, the garment is an undergarment and the capturing panel is no more than approximately 1 mm thick and is configured to receive at least approximately 0.25 tsp. of bodily fluid without reaching saturation. In other embodiments, the garment is an undergarment and the capturing panel is no more than approximately 2 mm thick and is configured to receive at least approximately 0.5 tsp. of bodily fluid without reaching saturation. In still other embodiments, the garment is an undergarment and said capturing panel is no more than approximately 3 mm thick and is configured to receive at least approximately 0.75 tsp. of bodily fluid without reaching saturation.

In one embodiment, the garment is a pair of low-coverage panties, for example, a thong. In another embodiment, the garment is a bra. In another embodiment, the garment is a shirt. In another embodiment, the garment is a pair of athletic shorts or pants. In another embodiment, the garment is a bib.

Some embodiments of the present invention provide a pair of washable low-coverage panties having a main garment body configured to be worn by a wearer comprising a crotch portion and a capturing panel attached to at least a portion of the crotch portion of the main garment body and comprising a receiving surface configured to interface with the crotch of the wearer and receive bodily fluids excreted from the wearer, wherein the capturing panel comprises a natural material, has a thickness no greater than approximately 4 mm, and is configured to transfer bodily fluid received from the wearer away from the receiving surface.

In some embodiments, the capturing panel includes a backing surface configured to interface with the main garment body and the capturing panel is constructed out of a wicking fabric configured to transfer received bodily fluid from the receiving surface to the backing surface. In some embodiments, the wicking fabric comprises a combination of wicking fibers and absorbing fibers, wherein the wicking fibers are concentrated about the receiving surface and the absorbing fibers are concentrated about the backing surface. In some embodiments, the natural material comprises plant or cellulose fibers, such as a 100% cotton fabric. The capturing may be attached to the main garment body by stitching only at substantially the perimeter of said capturing panel.

According to some embodiments, a fluid-resistant treatment may be disposed on at least a second portion of the main garment body. The second portion of the main garment body may comprise the first portion of the main garment body or may be the same as the first portion of the main garment body. In some embodiments, an odor-neutralizing treatment is applied to at least a portion of the capturing panel.

According to some embodiments, the capturing panel is no more than approximately 1 mm. thick and is configured to receive at least approximately 0.25 tsp. of bodily fluid without reaching saturation. In other embodiments, the capturing panel is no more than approximately 2 mm thick and is configured to receive at least approximately 0.5 tsp. of bodily fluid without reaching saturation. In still other embodiments, the capturing panel is no more than approximately 3 mm thick and is configured to receive at least approximately 0.75 tsp. of bodily fluid without reaching saturation.

### BRIEF DESCRIPTION OF THE SEVERAL VIEWS OF THE DRAWINGS

Having thus described embodiments of the invention in general terms, reference will now be made to the accompanying drawings, which are not necessarily drawn to scale, and wherein:

FIG. 1A is a perspective view of a garment in accordance with one embodiment of the present invention;

FIG. 1B is a top view of the garment of FIG. 1A;

FIG. 1C is an exploded view of the garment of FIG. 1A, illustrating how the capturing panel is incorporated into the garment;

FIG. 2A is a perspective view of a garment in accordance with another embodiment of the present invention;

FIG. 2B is an exploded view of the garment of FIG. 2A, illustrating how the capturing panel is incorporated into the garment;

FIG. 3A is a perspective view of a garment in accordance with another embodiment of the present invention;

FIG. 3B is a partially exploded view of the garment of FIG. 3A, illustrating how the capturing panels are incorporated into the garment;

FIG. 4A is a plan view of a garment in accordance with another embodiment of the present invention;

FIG. 4B is an cross-sectional view of the garment of FIG. 4A, illustrating how the capturing panels are incorporated into the garment; and

FIG. 5 is a cross-sectional view of a garment for capturing bodily fluids, according to one embodiment of the present invention.

### DETAILED DESCRIPTION OF EMBODIMENTS OF THE INVENTION

Embodiments of the present invention will now be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

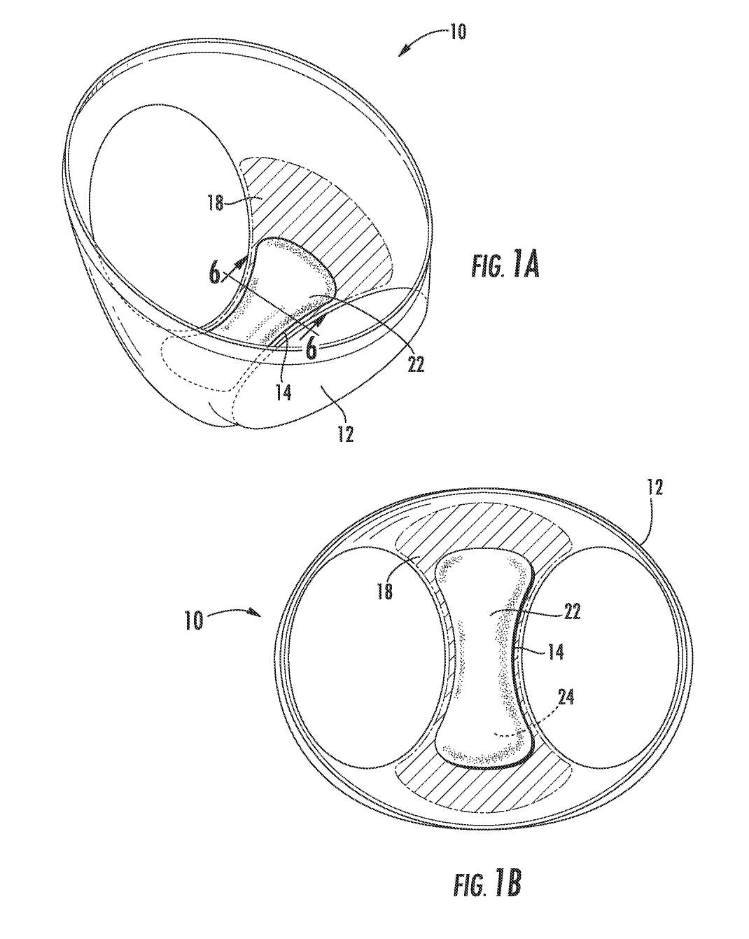

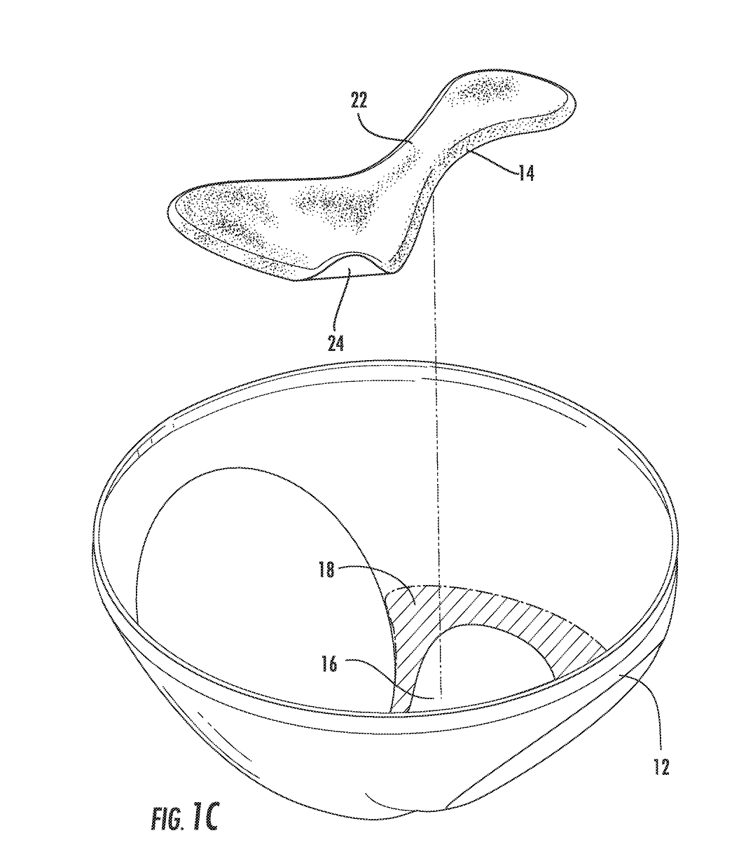

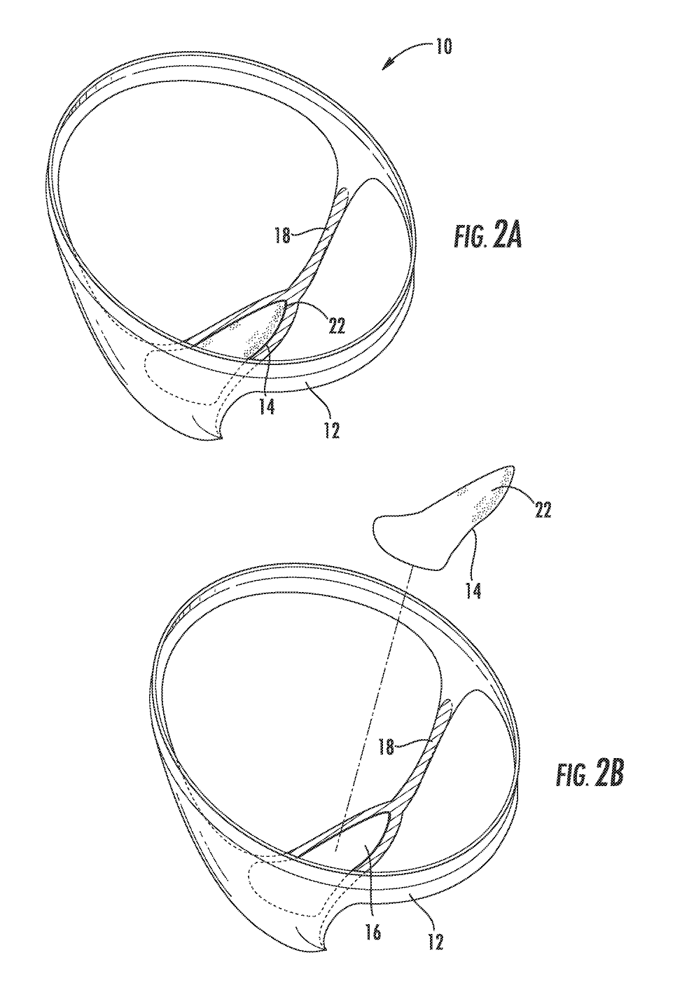

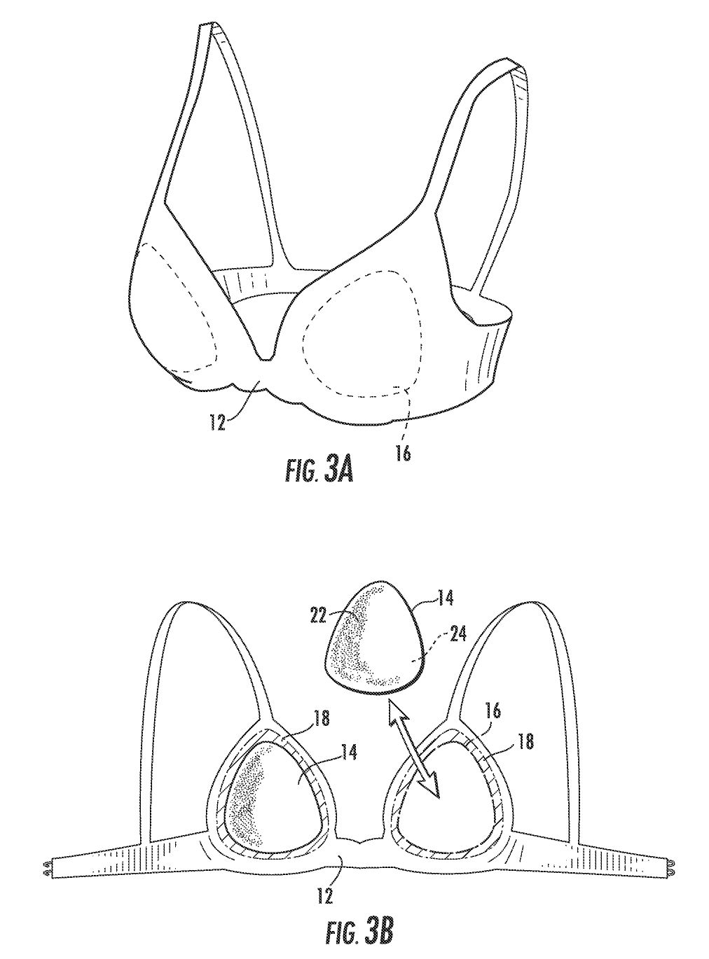

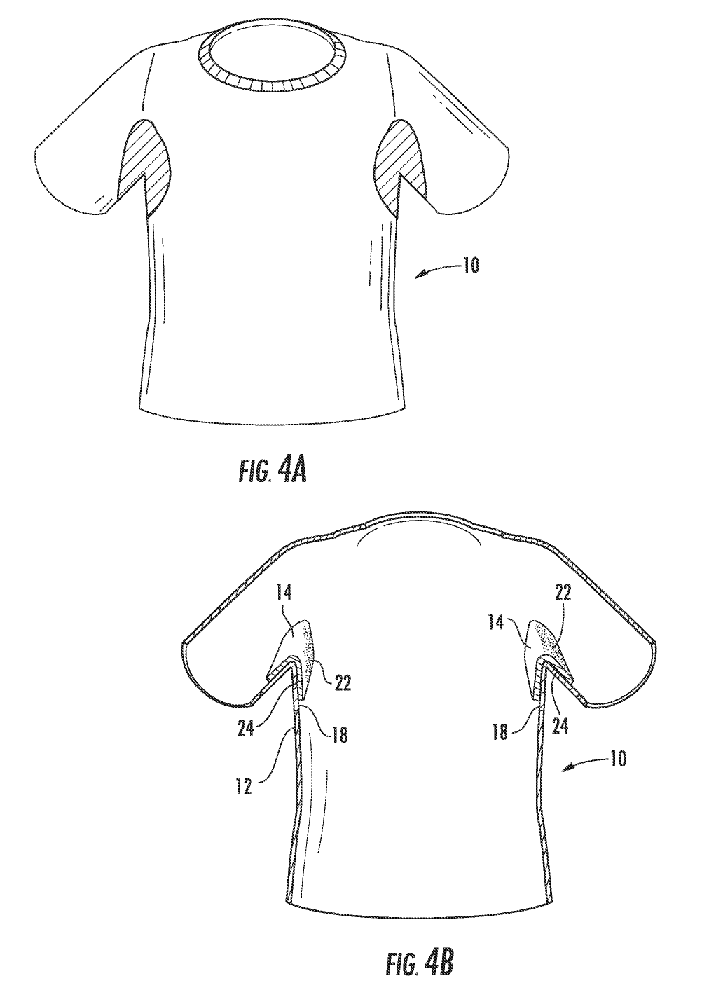

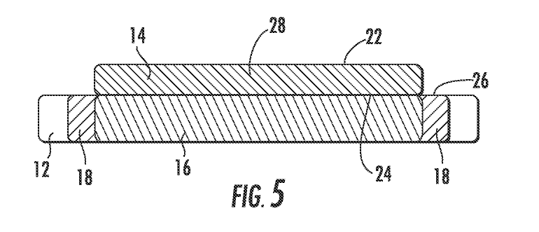

Referring to FIGS. 1A, 2A, 3A, and 4A, garments 10 in accordance with embodiments of the present invention are provided. The garment 10 may be any piece of wearable clothing. For example, as illustrated in FIG. 1A, the garment 10 may be a brief or bikini-style undergarment. As another example, as illustrated in FIG. 2A, the garment 10 may be a thong-style undergarment. As another example, as illustrated in FIG. 3A, the garment 10 may be a bra. As another example, as illustrated in FIG. 4A, the garment 10 may be a shirt. In other embodiments, the garment may be pants or shorts, such as yoga or other athletic pants or shorts, a long underwear top or bottom, pajama pants or shorts, any other type of women's underwear such as boy shorts, any other type of men's underwear such as boxers or boxer briefs, a piece of swimwear for men or women, or a bib, whether for an infant, child, or adult. In some embodiments, the garment 10 is a low-to-moderate coverage garment that is appropriate for daily wear. For example, according to some embodiments, the garment 10 is a pair of low-coverage women's panties.

As used herein, “low-coverage panties” means panties having a low-rise cut such that the waist of the panties when worn sits an appreciable distance below the wearer's belly button, for example, 2 inches or more, where the panties provide substantially complete coverage of the wearer's genitals, pubic region, anus, and perineum and either provide less than substantially complete coverage of the wearer's buttocks or provide substantially complete coverage of the wearer's buttocks, but the sides of the panties that connect the front of the panties to the back of the panties are narrow, for example, less than approximately 3 inches wide. Thus, “low-coverage panties” includes many types of thong and bikini style undergarments. Low-coverage panties are often a preferred choice among women both because of their perceived attractiveness and because they allow women to wear more revealing or tightly fitting clothing (such as low-rise pants or clingy dresses) without the panties becoming exposed or the shape of the panties being visible to others through the outer clothing. By providing low-coverage panties that have adequate protection against leaks and drips experienced by the wearer, embodiments of the present invention are a significant advancement over the bulky and full-coverage pads and undergarments of the prior art.

It should be understood that the foregoing examples are not exhaustive of the form that the garment 10 may take in accordance with different embodiments of the present invention.

As illustrated in FIGS. 1A, 1B, 2A, 3A, and 4A, the garment 10 comprises a main garment body 12 and at least one capturing panel 14. According to some embodiments, the main garment body 12 is an article constructed out of a fabric material and configured to be worn by a wearer. For example, as illustrated in FIG. 1A, the main garment body 12 is a brief or bikini-style undergarment. As another example, as illustrated in FIG. 2A, the main garment body 12 is a thong-style undergarment. As another example, as illustrated in FIG. 3A, the main garment body 12 is a bra. As another example, as illustrated in FIG. 4A, the main garment body 12 is a shirt. It should be understood that the foregoing examples are not exhaustive of the form that the main garment body 12 may take in accordance with different embodiments of the present invention. The main garment body 12 may be constructed from a single piece of fabric, or may be constructed from multiple pieces of material that are sewn or otherwise attached to each other. According to different embodiments, the material used to construct the main garment body 12 may be any type of washable fabric, including fabrics created from natural or synthetic fibers, textured fabric, including lace, treated fabric, a combination of the foregoing, etc.

According to some embodiments, the capturing panel 14 is constructed out of washable fabric material and is configured to receive and retain bodily fluid excreted by a wearer of the garment 10. As illustrated in FIG. 1B, the capturing panel 14 comprises a receiving surface 22 and a backing surface 24 opposite the receiving surface 22. The receiving surface 22 is configured to receive bodily fluids excreted by the wearer of the garment 10 and the backing surface 24 is configured to interface with the main garment body 12. The capturing panel 14 may be constructed from a single piece of fabric, or may be constructed from multiple pieces of fabric that are sewn or otherwise attached to each other. In addition, according to different embodiments, the capturing panel 14 may comprise only one layer of a single fabric or a combination of fabrics, or it may comprise multiple layers of one or more fabrics or combinations of fabrics. According to different embodiments, the fabric or fabrics used to construct the capturing panel 14 may be any type of washable fabric, including fabrics created from natural or synthetic fibers, textured fabric, treated fabric, a combination of the foregoing, etc.

In some embodiments, the material used to construct the capturing panel 14 and/or the main garment body 12 comprises a fabric created from natural fibers to maximize the breathability of the capturing panel for hygienic and comfort purposes. As used herein, “natural fibers” mean fibers made from plant, animal and mineral sources, as well as fibers made from regenerated cellulose, and a “natural material” means a fabric created from at least some natural fibers. In some embodiments, the natural material used to construct the capturing panel 14 comprises a fabric created from plant fibers or cellulose fibers, such as cotton or rayon, respectively. The natural material may not be comprised of 100% natural fibers. According to some embodiments, the natural material comprises between 50% and 100% natural fibers. According to some embodiments, the natural material comprises between 50% and 100% plant fibers or cellulose fibers. In some embodiments, the natural material is a 100% cotton fabric. In some embodiments, the natural material is a microfiber fabric comprising more than 50% plant fibers.

According to some embodiments, the capturing panel 14 has a surface area of approximately 8 in.², a thickness of approximately 1 mm., and is able to retain approximately 0.25 tsp. of bodily fluid while remaining dry on the surface of the capturing panel receiving the bodily fluid. In some embodiments, the just-described capturing panel 14 will not retain more than approximately 0.75 tsp. without reaching saturation. According to other embodiments, the capturing panel 14 has a surface area of approximately 8 in.², a thickness of approximately 2 mm, and is able to retain approximately 0.5 tsp. of bodily fluid while remaining dry on the surface of the capturing panel receiving the bodily fluid. In some embodiments, the just-described capturing panel 14 will not retain more than approximately 1 tsp. without reaching saturation. In other embodiments, the capturing panel 14 has a surface area of approximately 8 in.², a thickness of approximately 3 mm, and is able to retain approximately 0.75 tsp. of bodily fluid while remaining dry on the surface of the capturing panel receiving the bodily fluid. In some embodiments, the just-described capturing panel 14 will not retain more than approximately 1.5 tsp. without reaching saturation. Thus, for those embodiments where the garment 10 is an undergarment receiving excreted urine, the capturing panel 14 may have different thicknesses in order to provide different levels of protection to the wearer. For example, where the wearer experiences drips or leaks that are generally 0.25 tsp or less, a garment 10 incorporating the 1 mm thick capturing panel 14 may be preferable. On the other hand, a wearer experiencing leaks closer in volume to 0.5 tsp. on average may prefer the garment 10 incorporating the 2 mm thick capturing panel 14. According to some embodiments, the capturing panel 14, despite its minimum thickness, is effective in addressing such excretions because the capturing panel will be allowed to dry in between drips and leaks, as described in detail below.

According to some embodiments, the fabric used to construct the capturing panel is capable of retaining at least approximately 1/32 tsp. of fluid per square inch without reaching saturation and has a thickness no greater than approximately 4 mm. For example, in some embodiments, the fabric is capable of retaining at least approximately 1/32 tsp. of fluid per square inch and at most approximately 3/24 tsp. of fluid per square inch (this upper level being the point at which it reaches saturation) and has a thickness no greater than approximately 1 mm. According to other embodiments, the fabric is capable of retaining at least approximately 1/16 tsp. of fluid per square inch and at most approximately ⅛ tsp. of fluid per square inch and has a thickness no greater than approximately 2 mm. According to still other embodiments, the fabric is capable of retaining at least approximately 3/24 tsp. of fluid per square inch and at most approximately 3/16 tsp. of fluid per square inch and has a thickness no greater than approximately 3 mm.

In some embodiments, the capturing panel 14 comprises a single layer of material that is no more than approximately 4 mm thick. Furthermore, in some embodiments, the capturing panel 14 does not expand an appreciable amount in thickness when it is retaining excreted bodily fluid. Thus the capturing panel 14 will be a very subtle inclusion in the garment 10 and will not cause the wearer to suffer discomfort and embarrassment as the bulky absorbent pads of the prior art do. Further, because of its low-profile nature, the garment 10 does not need to be constructed to support a heavy, loaded pad, and therefore may be fashionable and low-coverage, such as low-coverage panties, allowing the wearer to wear form-fitting or more-revealing clothing over the garment 10.

In some embodiments, the material used to construct the capturing panel 14 is a fabric having wicking properties that provide for the transfer of fluid received by the fabric through the thickness of the fabric and/or across the area of the fabric. For example, the fabric may be configured to have one-way moisture transfer properties that cause the transfer of fluid that enters the fabric in one direction through the fabric. In some embodiments, the capturing panel 14 is constructed out of a fabric comprising both wicking and absorbing fibers that are woven in a manner such that the wicking fibers are concentrated toward one surface of the fabric and the absorbing fibers are concentrated toward the opposing surface of the fabric. As used herein, a wicking fiber means a fiber configured to transfer moisture along its surface and an absorbing fiber means a fiber configured to absorb moisture. A wicking fiber may be more adsorbent than absorbent. However, a wicking fiber may be capable of absorbing some moisture. In some embodiments, the wicking fiber is a natural fiber that has been treated in order to obtain its wicking properties. In some embodiments, the wicking fiber is a synthetic fiber. In some embodiments, the absorbent fiber is an untreated natural fiber. For example, the wicking fiber may be 100% cotton fiber that is treated in such a way to increase its wicking ability and absorbency, while decreasing its absorbency, while the absorbing fiber may be an untreated 100% cotton fiber. Alternatively, in some embodiments, the absorbent fiber may be a natural fiber treated to increase or otherwise adjust its absorbent properties.

In some embodiments, the wicking fibers within the material of the capturing panel 14 operate to transfer fluid and moisture through the fabric to neighboring wicking and absorbing fibers. According to some embodiments, wicking fibers are interspersed between the absorbing fibers, even in the region of the fabric having a concentration of absorbing fibers. This unique combination of fibers causes fluid and moisture to be transferred through the fabric away from the surface of the fabric having the concentration of wicking fibers and toward the surface of the fabric having the concentration of absorbing fibers, but not in the opposite direction. Furthermore, due to the interspersed wicking fibers, the fluid will also be transferred throughout the area of the fabric, not just through the thickness.

According to some embodiments the surface of the fabric having the concentration of absorbing fibers has a significantly lower wetting time and significantly higher absorption rate, maximum wetted radius, and spreading speed than the surface of the fabric having the concentration of wicking fibers. For example, in one embodiment, the surface of the fabric having the concentration of absorbing fibers has a wetting time of 3.478 seconds, an absorption rate of 56.047 percent per second, a maximum wetted radius of 20 mm, and a spreading speed of 3.538 mm per second, while the surface of the fabric having the concentration of wicking fibers has a wetting time of 10.597 seconds, an absorption rate of 12.347 percent per second, a maximum wetted radius of 13 mm, and a spreading speed of 1.269 mm per second. As another example, in one embodiment, the surface of the fabric having the concentration of absorbing fibers has a wetting time of 4.117 seconds, an absorption rate of 56.108 percent per second, a maximum wetted radius of 20 mm, and a spreading speed of 3.086 mm per second, while the surface of the fabric having the concentration of wicking fibers has a wetting time of 9.807 seconds, an absorption rate of 19.387 percent per second, a maximum wetted radius of 5 mm, and a spreading speed of 1.000 mm per second. It should be understood that the foregoing values are for exemplary purposes only, and the wetting time, absorption rate, wetted radius and spreading speed of both surfaces of the material may vary according to different embodiments.

By utilizing a natural material comprising a combination of wicking and absorbing fibers as described above to construct the capturing panel 14, the capturing panel 14 is configured to transfer any received bodily fluid received by the receiving surface 22 through the thickness of the capturing panel 14 toward the backing surface 24 and throughout the area of the capturing panel 14. Thus, the capturing panel 14 promotes the dryness of the receiving surface 22, which, in situations where the receiving surface 22 contacts the body of the wearer, for example, where the garment 10 is an undergarment, bra, or shirt as illustrated in FIGS. 1A, 2A, 3A, and 4A, promotes the comfort of the wearer by keeping the wearer dry and reducing the cling and chafing issues associated with wet fabric contacting the body, even if the backing surface 24 is saturated or damp. Furthermore, by transferring any fluid that enters the capturing panel 14 in a particular location into a larger area, the capturing panel disperses the received bodily fluid and avoids localized saturation at the point of entry. Thus, the capturing panel 14 both minimizes the likelihood of localized saturation of bodily fluids within the capturing panel 14, which in turn minimizes leaks out of the capturing panel 14. In addition, it promotes faster drying time of the capturing panel 14, as the reduced saturation allows the fluid to evaporate out of the capturing panel 14 faster.

Thus, the wicking properties of the material used to construct the capturing panel 14 offer a unique and improved solution to capturing relatively small amounts of bodily fluids excreted by a wearer of the garment 10 without allowing leaks and while promoting the dryness of the receiving surface 22 and increased overall drying times of the capturing panel 14, and thus, the comfort of the wearer. According to some embodiments, the material is a natural fabric made from natural plant fibers that have been treated to have increased wicking capabilities, and necessarily reduced absorbency, as well as fibers that have standard absorbency for their type of fiber. For example, the fabric may be a 100% cotton fabric woven from both cotton fibers treated with a wicking agent and untreated cotton fibers. Therefore, the capturing panel 14 may be constructed of a fabric that is actually treated to be less absorbent than it is prior to treatment. However, while the reduced absorbency of the fabric does necessarily constrain the overall amount of fluid that can be retained by the capturing panel 14, because, according to some embodiments, the garments 10 of the present invention are intended to be used by wearers to address bodily fluid excretions that do not exceed approximately 0.25 tsp. to 0.75 tsp. of fluid per excretion, rather than conditions such as moderate incontinence, the wicking properties of the capturing panel 14 are effective in ensuring that the bodily fluid excreted by the wearer is maintained within the capturing panel without leaking (provided the capturing panel 14 is configured to have a large enough area based on its absorbency per square inch to hold approximately 0.25 tsp. to 0.75 tsp. of bodily fluid throughout the capturing panel without reaching saturation) and offer the advantages of increased drying times and a drier receiving surface.

According to some embodiments, and as illustrated in FIGS. 1B, 2B, 3B, 4B, and 5B, the capturing panel 14 is attached to the main garment body 12 such that the capturing panel 14 covers at least a first portion 16 of the main garment body 12. The first portion 16 of the main garment body 12 that is covered by the capturing panel 14 may be any portion of the main garment body 12 that would be likely to receive bodily fluids from the wearer of the garment 10 in the absence of the capturing panel 14. For example in the case of an undergarment such as low-coverage panties, as illustrated in FIGS. 1B and 2B, the capturing panel 14 is attached to the main garment body 12 such that the capturing panel covers a first portion 16 of the main garment body 12 comprising an interior crotch portion of the main garment body 12. This interior crotch portion is the portion of the main garment body 12 that would interface with the crotch of the wearer of the garment 10 in the absence of the capturing panel 14. Thus, the capturing panel 14 is attached to the main garment body 12 such that it will receive bodily fluids that would otherwise enter the interior crotch portion of the main garment body 12, such as urine, menstrual fluid, and vaginal discharge. As another example, and as illustrated in FIG. 3B, the first portion 16 of the main garment body 12 comprises an interior cup portion of the main garment body 12 that would interface with a nipple of the wearer of the garment 10 in the absence of the capturing panel 14. Thus, the capturing panel 14 is attached to the main garment body 12 such that it will receive bodily fluids that would otherwise enter the interior cup portion of the main garment body 12 from the nipple, such as breast milk. As yet another example, and as illustrated in FIG. 4B, the first portion 16 of the main garment body 12 comprises an underarm portion of the main garment body 12 that would interface with the underarms of the wearer of the garment 10 in the absence of the capturing panel 14. Thus, the capturing panel 14 is attached to the main garment body 12 such that it will receive bodily fluids that would otherwise enter the underarm portion of the main garment body 12, such as sweat.

According to one embodiment, the capturing panel 14 is sewn onto the main garment body 12, however, any other known method of attachment may be used, including fabric glue, fabric tape, etc. The capturing panel 14 may be sewn onto the main garment body 12 such that the perimeter of the capturing panel 14 is stitched down to the main garment body 12, but the interior of the capturing panel 14 is not attached to the first portion 16 of the main garment body 12 that the capturing panel 14 covers. This is advantageous as it allows air circulation in between the capturing panel and the first portion 16 of the main garment body 12, which promotes evaporation of any fluid retained by the capturing panel 14 for faster drying times.

According to some embodiments of the present invention, in addition to the capturing panel 14, a fluid-resistant treatment or layer is provided in the garment 10 that is configured to prevent any fluid captured by the capturing panel 14 from penetrating the main garment body 12. For example, a fluid-resistant treatment may be applied to the backing surface 24 of the capturing panel 14 or on the surface of the main garment body 12 that interfaces with the backing surface 24 of the capturing panel 14. In this way, a barrier is constructed to prevent bodily fluid from penetrating the main garment body 12. However, according to some embodiments, the fluid resistant treatment does not substantially hinder the breathability of the garment 10. For example, where the fluid-resistant treatment is applied to the main garment body 12, the main garment body remains breathable such that air and water vapor can pass through the material of the main garment body 12. This advantageously allows the bodily fluid in the capturing panel 14 to evaporate, drying the capturing panel 14.

Thus, unlike the absorbent products of the prior art that receive and encapsulate or otherwise hold urine or other bodily fluid until the wearer can replace the product, garments 10 in accordance with embodiments of the present invention are configured to receive bodily fluid into the capturing panel 14, disperse the bodily fluid throughout the capturing panel 14 and away from the receiving surface 22 to avoid localized saturation, promote the elimination of the bodily fluid from the capturing panel 14 through evaporation without allowing penetration of the bodily fluid to the outer surface of the garment 10, and, upon elimination of the bodily fluid from the capturing panel 14, have capacity to receive more bodily fluid into the capturing panel 14. The foregoing novel aspects of embodiments of the present invention, i.e. the wicking capabilities of the capturing panel 14 combined with the breathability of the garment 10 for evaporation, allow the garment 10 to be low-profile and low-coverage while still being capable of effectively handling numerous excretions of bodily fluid over a period of time, including excretions that would together amount to a greater volume of fluid than the capturing panel 14 is configured to retain at one time.

According to different embodiments, the fluid-resistant treatment utilized may include the application of a laminate, coating, or durable water repellant treatment to the fabric of the main body portion 12. For example, according to different embodiments, the fluid-resistant treatment may include a porous fluoropolymer membrane coating or a finishing treatment. The fluid-resistant treatment may be applied through a bath or by spray-on or foam applications. According to one embodiment, the fluid-resistant treatment comprises a durable water repellant treatment that includes subjecting the fabric of the main body portion 12 to a nanoparticle C6 fluorochemical finishing bath. The nanoparticles form a fine structure on the surface of the fabric that prevents fluid and other substances from adhering. Fabric treated in this manner will not only be fluid-resistant, but as an added advantage, will also be resistant to stains. Further, the treated fabric is breathable and will maintain its resistance to fluid even after multiple washings of the garment 10. As an alternative to a fluid-resistant treatment of the main body portion 12, in some embodiments of the present invention, a fluid-resistant material is disposed between the capturing panel 14 and the main garment body 12 to prevent the transfer of bodily fluid from the capturing panel to the main garment body 12. For example, a thin layer of chemically treated fabric, rubber, or other fluid-resistant material may be attached to the main garment body 12 to protect the main garment body 12 from any leakage from the capturing panel 14.

According to one embodiment, a breathable fluid-resistant treatment is applied to at least a second portion 18 of the main garment body 12. According to different embodiments, the second portion 18 that is treated by the fluid-resistant treatment may be the same as the first portion 16 of the main garment body 12 that is covered by the capturing panel 14, or may be different from the first portion 16. According to different embodiments, the second portion 18 of the main garment body 12 may totally encompass the first portion 16 such that the entire first portion 16 is treated with the fluid-resistant treatment, whether the second portion 18 is larger than the first portion 16 or the same as the first portion 16, or the second portion 18 may merely overlap the first portion 16 such that certain portions of the first portion 16 are not treated. According to some embodiments, the first portion 16 of the main garment body 12 is treated with the breathable fluid-resistant treatment such that the backing surface 24 of the capturing panel 14 interfaces with the treated surface of the main garment body 12. Thus, any bodily fluid that collects on the backing surface of the capturing panel 14 is prevented from penetrating the main garment body 12. This prevents any other clothing that contacts the main garment body 12, for example, exterior clothing in the event the garment 10 is worn as an undergarment, from contacting any wet surface of the garment 10. In some embodiments, the breathable fluid-resistant treatment applied will not be eliminated or substantially lose effectiveness during or following multiple washings of the garment 10. In one embodiment, the treatment is not eliminated and maintains a level of effectiveness throughout the life of the garment 10 (where the “life” of a garment means the appropriate number of washings before the garment is no longer in good condition).

According to some embodiments, the garment 10 may also include an odor-neutralizing treatment configured to neutralize, absorb and/or eliminate any odors attendant to the bodily fluids captured by the capturing panel 14. The odor treatment may be applied to any portion of the capturing panel 14 and/or the main garment body 12. In one embodiment, the odor treatment is applied to the entire capturing panel 14. In another embodiment, the odor treatment is applied to the entire capturing panel 14 and the entire main garment body 12. In some embodiments, the odor-neutralizing treatment applied will not be eliminated or substantially lose effectiveness during or following multiple washings of the garment 10. In one embodiment, the treatment is not eliminated and maintains a level of effectiveness throughout the life of the garment 10.

According to one embodiment, the odor treatment method utilized comprises chemically bonding an odor-encapsulating agent to the capturing panel 14 and/or the main garment body 12, reacting a cyclodextrin with a cross-linking agent capable of forming ether bonds with the cyclodextrin and with the capturing panel 14 and/or the main garment body 12, and curing the textile material treated with a mixture of the cyclodextrin and the cross-linking agent. In such embodiments, the cross-linking agent utilized may be imidazolidone, which forms an ether bond with a hydroxyl group on the cyclodextrin and with a hydroxyl group. In the event the capturing panel 14 and/or the main garment body 12 is constructed out of a fabric containing cellulose, imidazolidone forms an ether bond with a hydroxyl group on the cellulose. Therefore, according to some embodiments, it is advantageous to construct one or both of the capturing panel 14 and the main garment body 12 out of a fabric containing cellulose, such as a fabric made from plant fibers or cellulose fibers, and apply the just-described odor treatment to either or both the capturing panel 14 and the main garment body 12, or portions thereof. The fabric utilized may be a fabric that is 100% comprised of plant or cellulose fibers, or it may be comprised of less than 100% plant or cellulose fibers. In some embodiments, the fabric is a fabric that is between 50-100% comprised of plant or cellulose fibers. While the foregoing odor-neutralizing treatment method had been described, it should be understood that any odor treatment known by those skilled in the art may be utilized to treat the garment 10.

Referring now to FIG. 6, a cross-sectional view of the garment 10 taken along line 6-6 as shown in FIG. 1A is provided. As illustrated, the capturing panel 14 is configured such that the receiving surface 22 of the capturing panel 14 receives bodily fluid excreted by the wearer of the garment 10 and the backing surface 24 of the capturing panel 14 interfaces with the first portion 16 of the main garment body 12. As discussed above, the first portion 16 of the main garment body 12 may be treated with a fluid-resistant treatment 26 that is applied to the second portion 18 of the main garment body 12 that includes the first portion 16. Further, the capturing panel 14 may be treated with an odor treatment 28. The bodily fluid enters the garment 10 through the receiving surface 22 of the capturing panel 14. The fabric of the capturing panel 14 serves to transfer the fluid received away from the receiving surface 22 and across the capturing panel 14 to avoid localized saturation. In the event there is any saturation, or any fluid collects on the backing surface 24 of the capturing panel 14, the fluid-resistant treatment applied to the main garment body 12 prevents the fluid from penetrating the main garment body. Further, any odor emanating from the bodily fluid will be minimized by the odor treatment applied to the capturing panel 14.

Specific embodiments of the invention are described herein. Many modifications and other embodiments of the invention set forth herein will come to mind to one skilled in the art to which the invention pertains having the benefit of the teachings presented in the foregoing descriptions and the associated drawings. Therefore, it is to be understood that the invention is not to be limited to the specific embodiments disclosed and that modifications and other embodiments and combinations of embodiments are intended to be included within the scope of the appended claims. Although specific terms are employed herein, they are used in a generic and descriptive sense only and not for purposes of limitation.

What is claimed is:

1. A washable garment, the garment comprising:
a main garment body configured to be worn by a wearer; and
a capturing panel comprising a receiving surface configured to receive bodily fluids excreted from the wearer attached to said main garment body such that said capturing panel covers at least a first portion of said main garment body,
wherein said capturing panel comprises a natural material, has a thickness no greater than approximately 4 mm, and is configured to transfer bodily fluid received from the wearer away from the receiving surface.

2. The garment of claim 1, wherein said capturing panel comprises a backing surface configured to interface with the main garment body and wherein said capturing panel is constructed out of a wicking fabric configured to transfer received bodily fluid from the receiving surface to the backing surface.

3. The garment of claim 2, wherein the wicking fabric comprises a combination of wicking fibers and absorbing fibers, wherein the wicking fibers are concentrated about the receiving surface and the absorbing fibers are concentrated about the backing surface.

4. The garment of claim 1, wherein the natural material comprises plant or cellulose fibers.

5. The garment of claim 1, wherein the natural material is a 100% cotton fabric.

6. The garment of claim 1, wherein said capturing panel is attached to said main garment body by stitching only at substantially the perimeter of said capturing panel.

7. The garment of claim 1, wherein a fluid-resistant treatment is disposed on at least a second portion of said main garment body.

8. The garment of claim 7, wherein the at least a second portion of said main garment body comprises the at least a first portion of said main garment body.

9. The garment of claim 7, wherein the at least a second portion of said main garment body is the same as the at least a first portion of said main garment body.

10. The garment of claim 7, wherein the fluid-resistant treatment does not substantially reduce the breathability of said main garment body.

11. The garment of claim 7, wherein the fluid-resistant treatment comprises a nanoparticle C6 fluorochemical treatment configured to reduce the ability of fluids and substances to adhere to or penetrate said main garment body.

12. The garment of claim 1, wherein an odor-neutralizing treatment is applied to at least a portion of said capturing panel.

13. The garment of claim 12, wherein the capturing panel comprises plant or cellulose fibers and the odor neutralizing treatment comprises bonding an odor-encapsulating agent to the capturing panel, reacting a cyclodextrin with a cross-linking agent capable of forming ether bonds with the cyclodextrin and with the capturing panel, and curing the textile material treated with a mixture of the cyclodextrin and the cross-linking agent

14. The garment of claim 1, wherein the garment is an undergarment and said capturing panel is no more than approximately 1 mm thick and is configured to receive at least approximately 0.25 tsp. of bodily fluid without reaching saturation.

15. The garment of claim 1, wherein the garment is an undergarment and said capturing panel is no more than approximately 2 mm thick and is configured to receive at least approximately 0.5 tsp. of bodily fluid without reaching saturation.

16. The garment of claim 1, wherein the garment is an undergarment and said capturing panel is no more than approximately 3 mm thick and is configured to receive at least approximately 0.75 tsp. of bodily fluid without reaching saturation.

17. The garment of claim 1, wherein the garment is a pair of low-coverage panties.

18. The garment of claim 17, wherein the garment is a thong.

19. The garment of claim 1, wherein the garment is a bra.

20. The garment of claim 1, wherein the garment is a shirt.

21. The garment of claim 1, wherein the garment is a pair of athletic shorts or pants.

22. The garment of claim 1, wherein the garment is a bib.

23. A pair of washable low-coverage panties, the panties comprising:
a main garment body configured to be worn by a wearer comprising an interior crotch portion; and
a capturing panel comprising a receiving surface configured to receive bodily fluids excreted from the wearer attached to said main garment body such that said capturing panel covers at least a portion of the interior crotch portion of said main garment body,
wherein said capturing panel comprises a natural material, has a thickness no greater than approximately 4 mm, and is configured to transfer bodily fluid received from the wearer away from the receiving surface.

24. The panties of claim 23, wherein said capturing panel comprises a backing surface configured to interface with the main garment body and wherein said capturing panel is constructed out of a wicking fabric configured to transfer received bodily fluid from the receiving surface to the backing surface.

25. The panties of claim 24, wherein the wicking fabric comprises a combination of wicking fibers and absorbing fibers, wherein the wicking fibers are concentrated about the receiving surface and the absorbing fibers are concentrated about the backing surface.

26. The panties of claim 23, wherein the natural material comprises plant or cellulose fibers.

27. The panties of claim 23, wherein the natural material is a 100% cotton fabric.

28. The panties of claim 23, wherein said capturing panel is attached to said main garment body by stitching only at substantially the perimeter of said capturing panel.

29. The panties of claim 23, wherein a fluid-resistant treatment is disposed on at least a second portion of said main garment body.

30. The panties of claim 29, wherein the at least a second portion of said main garment body comprises the at least a first portion of said main garment body.

31. The panties of claim 29, wherein the at least a second portion of said main garment body is the same as the at least a first portion of said main garment body.

32. The panties of claim 23, wherein an odor-neutralizing treatment is applied to at least a portion of said capturing panel.

33. The panties of claim 23, wherein said capturing panel is no more than approximately 1 mm thick and is configured to receive at least approximately 0.25 tsp. of bodily fluid without reaching saturation.

34. The panties of claim 23, wherein said capturing panel is no more than approximately 2 mm thick and is configured to receive at least approximately 0.5 tsp. of bodily fluid without reaching saturation.

35. The panties of claim 23, wherein said capturing panel is no more than approximately 3 mm thick and is configured to receive at least approximately 0.75 tsp. of bodily fluid without reaching saturation.
